(19) United States
(12) Patent Application Publication
(10) Pub. No.: US 2013/0236972 A1
(43) Pub. Date: Sep. 12, 2013

(54) Liver Sinusoid Model

(71) Applicants: Hongseok Noh, Ambler, PA (US); Michael Bouchard, Philadelphia, PA (US)

(72) Inventors: Hongseok Noh, Ambler, PA (US); Michael Bouchard, Philadelphia, PA (US)

(73) Assignees: PHILADELPHIA HEALTH & EDUCATION CORPORATION D/B/A DREXEL UNIVERSITY COLLEGE OF MEDICINE, PHILADELPHIA, PA (US); DREXEL UNIVERSITY, PHILADELPHIA, PA (US)

(21) Appl. No.: 13/413,679

(22) Filed: Mar. 7, 2012

(51) Int. Cl. C12N 5/071; C12M 3/00

(52) U.S. Cl. 435/401; 435/289.1

(57) ABSTRACT

A liver sinusoid model includes a generally planar substrate having first and second generally parallel microchannels formed therein. A microporous membrane is disposed between and separating the first and second generally parallel microchannels. A first layer of cells lines one side of the membrane in the first microchannel. The first layer of cells are all a first common cell type. A second layer of cells extends parallel to the first layer of cells in one of the first microchannel and the second microchannel. The second layer of cells is all of a second common cell type. A liver sinusoid bioreactor utilizing the inventive model is also disclosed.

### STATEMENT REGARDING GOVERNMENT SPONSORED RESEARCH OR DEVELOPMENT

The present invention was developed under grant number 0747752, awarded by the National Science Foundation and grant number R21AA017458, awarded by the National Institutes of Health. The U.S. Government has certain rights in this invention.

### FIELD OF THE INVENTION

The present invention relates to a miniature liver tissue model that represents the architecture and functions of human liver tissue.

### BACKGROUND OF THE INVENTION

The liver is the largest solid organ in the body and is involved in a myriad of metabolic processes required for body homeostasis, as well as the detoxification of harmful chemicals. Hepatocytes are the major cells within the liver and are responsible for many activities that are attributed to the liver. Liver biology studies predominantly rely on hepatocyte culture models. When hepatocytes are isolated and cultured in vitro, however, they lose their normal structure and functions because of a lack of cell-to-cell and cell-to-extracurricular matrix interactions that are essential for maintaining normal liver functions. While much progress has been made in the past in prolonging hepatocyte viability and maintaining liver functions in vitro, there are still no authentic liver models that accurately represent the architecture and functions of human liver tissue.

### BRIEF SUMMARY OF THE INVENTION

Briefly, the present invention provides a liver sinusoid model comprising a generally planar substrate having first and second generally parallel microchannels formed therein. A microporous membrane is disposed between and separating the first and second generally parallel microchannels. A first layer of cells lines one side of the membrane in the first microchannel. The first layer of cells are all a first common cell type. A second layer of cells extends parallel to the first layer of cells in one of the first microchannel and the second microchannel. The second layer of cells is all of a second common cell type.

Further, the present invention provides a liver sinusoid functional unit comprising a generally planar substrate having first and second generally parallel microchannels formed therein. The first microchannel is disposed above the second microchannel. A microporous membrane is disposed between the first microchannel and the second microchannel. A layer of hepatocyte cells is disposed in the first microchannel and extends directly along the microporous membrane. A layer of liver sinusoidal endothelial cells is disposed in the first microchannel such that the layer of hepatocyte cells are sandwiched between the layer of liver sinusoidal endothelial cells (LSECs) and the microporous membrane

Additionally, the present invention provides bioreactor functions that include continuous perfusion of culture media and introduction of drugs. A first microchannel represents a sinusoid (blood vessel) and thus a fluid flow that simulates blood with proper oxygen and nutrient compositions is introduced. A second microchannel represents a duct of bile that is secreted from hepatocytes and transferred to the intestines, and thus a fluid flow will be introduced to collect the bile component.

### BRIEF DESCRIPTION OF THE DRAWINGS

The foregoing summary, as well as the following detailed description of the invention, will be better understood when read in conjunction with the appended drawings. For the purpose of illustrating the invention, there is shown in the drawing certain embodiments of the present invention. It should be understood, however, that the invention is not limited to the precise arrangements shown. In the drawings:

FIG. 1 is a side elevational view of a liver sinusoid model according to a first exemplary embodiment of the present invention;

FIG. 2 is a side elevational view of an enlarged portion of the liver sinusoid model of FIG. 1, showing flow through the first microchannel;

FIG. 3 is a schematic view of a mini liver bioreactor flow circuit incorporating the liver sinusoid model of FIG. 1; and

FIG. 4 is a side elevational view of a liver sinusoid model according to a second exemplary embodiment of the present invention.

### DETAILED DESCRIPTION OF THE INVENTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

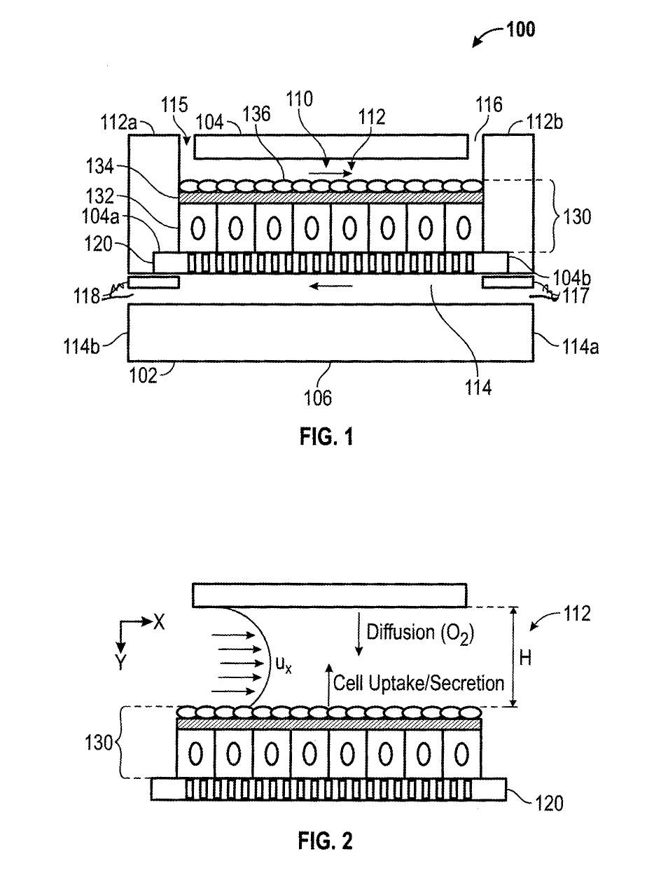

Referring to FIG. 1, a first exemplary embodiment of a liver sinusoid model 100 according to the present invention is shown. Liver sinusoid model 100 is an in vitro microfluidic model of a model of the liver tissue, a liver sinusoid. Liver sinusoid model 100 is formed using a generally planar substrate assembly 102 as a base. Substrate assembly 102 may be constructed from planar polymer sheets constructed from a material such as, for example, polymethyl methacrylate (“PMMA”) or polydimethylsiloxane (“PDMS”). PMMA and PDMS can be used because of the ease in forming microchannels therein. The processes of manufacturing microchannels in PMMA and PDMS substrates are well known by those skilled in the art. Substrate assembly 102 includes a top substrate 104 and a bottom substrate 106. A microchannel assembly 110 is formed in the substrates 104, 106. A first, or top, microchannel 112 is formed in top substrate 104 and a second, or bottom, microchannel 114 is formed in bottom substrate 106. First microchannel 112 is used to simulate a capillary that provides blood to liver sinusoid model 100. Second microchannel 114 is used to simulate a bile duct that removes toxins and other waste products from liver sinusoid model 100.

In an exemplary embodiment, planar substrate assembly 102 has dimensions of approximately 10-20 millimeters wide, 20-40 millimeters long, and 5-10 millimeters thick. Top and bottom microchannels, 112 and 114 have a length of about 10-20 millimeters, a width of about 1-2 millimeters, and a depth of about 50-200 microns. Top microchannel 112 also includes a top inlet passage 115 at a first end 112a of top microchannel 112 and a top outlet passage 116 and a second end 112b of top microchannel 112. Top inlet passage 115 and top outlet passage 116 each extend generally transverse to the length of top microchannel 112.

Bottom microchannel 112 also includes a bottom inlet passage 117 at a first end 114a of bottom microchannel 114 and a bottom outlet passage 118 at a second end 114b of bottom microchannel 114. A microporous membrane 120 is placed over the top of bottom substrate 106 so that membrane 120 covers microchannel 114. In a first exemplary embodiment, membrane 120 may be constructed from a Transwell membrane (polyester) or parylene polymer (polyparaxylylene) that is about 10 microns thick. Pores in membrane 120 may be between about 0.3 and about 1 micron in diameter. It is desired that the pores are sufficiently large enough to allow liquids and proteins to pass through from one side of membrane 120 to opposing side of membrane 120, yet small enough to prevent cells from passing through membrane 120. Top substrate 104 is placed on top of bottom substrate 106 and substrates 104, 106 are secured to each other so that second microchannel 114 is generally parallel to first microchannel 112, with microchannels 112, 114 being separated from each other by membrane 120. In an exemplary embodiment, top substrate 104 includes a first groove 104a and a second groove 104b that are sized to accept and retain membrane 120 between top substrate 104 and bottom substrate 106. Top substrate 104 is fixedly coupled to bottom substrate 106 via thermal fusion bonding or adhesive bonding or oxygen plasma, which welds top substrate 104 to bottom substrate 106.

In order to prepare first microchannel 112 to receive liver cells, a collagen solution is flushed through first microchannel 112 via top inlet passage 115 and out of top outlet passage 116. The collagen solution allows liver cells to adhere to membrane 120. A plurality of liver cells 130 are disposed on membrane 120 in first microchannel 112. In an exemplary embodiment, liver cells 130 may be rat liver cells. In an alternative exemplary embodiment, liver cells 130 may be human liver cells. Liver cells 130 include, extending outwardly from membrane 120, a layer of hepatocyte cells 132 directly on membrane 120, a collagen layer 134 directly on the layer of hepatocyte cells 132, and a layer of liver sinusoidal endothelial cells (LSEC) 136 directly on collagen layer 134. Included with the LSEC 136 are minority cells, such as stellate cells and Kupffer cells, which are liver-specific micro phages. In an exemplary embodiment, liver cells 130 are disposed on membrane 120 in the absence of any fibroblast cells. Those skilled in the art, however, will recognize that fibroblast cells may also be used to assist in culturing liver cells 130.

Liver cells 130 are primary cells, meaning that they are freshly removed from a recently deceased body, and are viable within liver sinusoid model 100 for a timeframe greater than at least one month. Collagen layer 134 simulates the Space of Disse, which separates hepatocyte cells from the LSEC in a biological liver. In this embodiment, all liver cells are located in first microchannel 112. Optionally, established cell lines such as rat adrenal medulla endothelial cells (RAMEC) can also be used to replace the LSECs.

Liver sinusoid model 100 can be used in a static mode. A first fluid, representing blood, may be inserted into first microchannel 112 via either top inlet passage 115 or top outlet passage 116. Additionally, a second fluid, representing bile fluid, may be inserted into second microchannel 114 via either bottom inlet passage 117 or bottom outlet passage 118. LSEC 136 and hepatocyte cells 132 act upon the fluid in first microchannel 112.

FIG. 2 illustrates an enlarged version of top microchannel 112 having a height “H” and showing fluid flow having a velocity uₓ along x and y axes. Oxygen diffuses from the first fluid, through liver cells 130 and membrane 120, while cell uptake/secretion from liver cells 130 is absorbed by the first fluid.

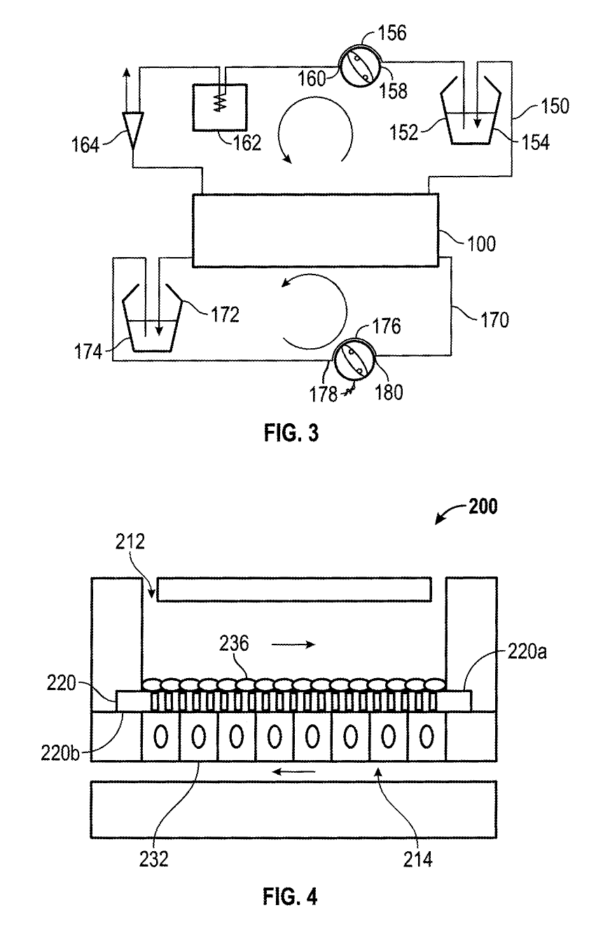

Alternatively, in an exemplary embodiment, illustrated schematically in FIG. 3, a continuous perfusion system can be provided to liver sinusoid model 100 to simulate the flow of blood to liver sinusoid model 100 as well as the discharge of bile from liver sinusoid model 100. The perfusion system provides bioreactor functions that include continuous perfusion of culture media and introduction of drugs. First microchannel 112 represents a sinusoid (blood vessel) and thus a fluid flow that simulates blood with proper oxygen and nutrient compositions is introduced. Second microchannel 114 represents a duct of bile that is secreted from hepatocytes and transferred to the intestines (not shown), and thus a fluid flow will be introduced to collect the bile component.

First microchannel 112 of liver sinusoid model 100 is in fluid communication with a first fluid circuit 150 that is used to provide continuous perfusion to simulate blood being pumped through first microchannel 112. First fluid circuit 150 represents a sinusoid (blood vessel) and includes a medium reservoir 152 that includes a fluid medium 154 that simulates blood. Fluid medium 154 is a formulation that includes growth factors, hormones, nutrients, and oxygen.

A peristaltic pump 156 includes a suction end 158 in fluid communication with fluid medium 154 and is used to pump fluid medium 154 from medium reservoir 152. In an exemplary embodiment, peristaltic pump 156 is a compact digital pump, manufactured by Ismatic. A discharge end 160 of peristaltic pump 156 is in fluid communication with a medium oxygenator 162 and pump fluid medium 154 into medium oxygenator 162. In an exemplary embodiment, medium oxygenator 162 is realized by passing the fluid through a PDMS tube in an oxygen-rich bottle.

A discharge end of medium oxygenator 162 is in fluid communication with a bubble trap 164, which is used to remove any air bubbles from fluid medium 154. In exemplary embodiment, bubble trap 164 consists of a micro-porous, hydrophobic membrane where an aqueous fluid is retained and able to flow from inlet to outlet while air bubbles are forced through to a vent.

A discharge end of bubble trap 164 is in fluid communication with top inlet passage 115 of first microchannel 112. Top outlet passage 116 of first microchannel 112 is in fluid communication with medium reservoir 152 such that fluid that flows into top inlet passage 115 of first microchannel 112 flows through first microchannel 112 and out of top outlet passage 116 of first microchannel 112 and back to medium reservoir 152. The flow rate of fluid medium 154 through first microchannel 112 can be controlled by adjusting the operational speed of peristaltic pump 156. Adjustment of the flow rate of fluid medium 154 through first microchannel 112 results in an adjustment of the oxygen concentration within fluid medium 154, as well as the shear stress imparted upon LSEC 136 by fluid medium 154 flowing across LSEC 136.

Additionally, second microchannel 114 is in fluid communication with a second fluid circuit 170 that is used to provide continuous perfusion to simulate bile fluid that is being pumped through second microchannel 114. Second fluid circuit 170 is adapted to collect waste product generated by liver cells 130 and includes a bile collection reservoir 172 that includes a fluid medium 174 that simulates bile fluid.

A peristaltic pump 176 includes a suction end 178 in fluid communication with fluid medium 174 and is used to pump fluid medium 174 from bile collection reservoir 172. A discharge end 180 of peristaltic pump 176 is in fluid communication with bottom inlet passage 117 of second microchannel 114. Bottom outlet passage 118 of second microchannel 114 is in fluid communication with bile collection reservoir 172 such that fluid that flows into bottom inlet passage 117 of second microchannel 114 flows through second microchannel 114 and out of bottom outlet passage 118 of second microchannel 114 and back to bile collection reservoir 172. The flow rate of fluid medium 174 through second microchannel 114 can be controlled by adjusting the operational speed of peristaltic pump 176. As shown in FIG. 2, the flow direction of fluid medium 154 through first microchannel 112 is in a left-to-right direction, such that fluid flow in first fluid circuit 150 is counterclockwise.

Referring now to FIG. 4, a second exemplary embodiment of a liver sinusoid model 200 according to the present invention is shown. Liver sinusoid model 200 may be formed using a generally planar substrate assembly 202 similar to generally planar substrate assembly 102 disclosed above. Structural elements in liver sinusoid model 200 are similar to structural elements in liver sinusoid model 100 but are identified with a “2” as the first digit in the reference number instead of a “1” as the first digit in the reference number.

Liver sinusoid model 200 is similar to liver sinusoid model 100 disclosed above, but with a structural modification. A difference between liver sinusoid model 100 and liver sinusoid model 200 is that, while, in liver sinusoid model 100, hepatocyte cells 132 and LSEC 136 are on the same side of membrane 120 in first microchannel 112, in liver sinusoid model 200, hepatocyte cells 232 are on an opposing side of a microporous membrane 220 in a second microchannel 214. In liver sinusoid model 200, LSEC 236 are attached directly to a first side 220a of membrane 220 while hepatocyte cells 232 are attached directly to a second side 220b of membrane 220. Similar to liver sinusoid model 100, a collagen solution is flushed through first microchannel 212 to assist in the adhesion of LSEC 236 to first side 220a of membrane 220 and a collagen solution is flushed through second microchannel 214 to assist in the adhesion of hepatocyte cell 232 to second side 220b of membrane 220. In liver sinusoid model 200, collagen layer 134 may be omitted. Similar to liver sinusoid model 100, liver sinusoid model 200 can be used in a static or a dynamic environment.

The liver functional model according to the present invention provides an accurate liver tissue model for performing liver biology studies, liver cancer research, and viral infections, as well as toxicology studies, drug metabolism studies, the effects of alcohol and viral infection on the liver, as well as performing drug screening tests.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A liver sinusoid model comprising:
a generally planar substrate having first and second generally parallel microchannels formed therein;
a microporous membrane disposed between and separating the first and second generally parallel microchannels;
a first layer of cells lining one side of the membrane in the first microchannel, wherein all of the cells in the first layer are a first common cell type; and
a second layer of cells extending parallel to the first layer of cells in one of the first microchannel and the second microchannel, wherein all of the cells in the second layer are a second common cell type.

2. The liver sinusoid model according to claim 1, wherein the first layer of cells comprises a layer of hepatocyte cells.

3. The liver sinusoid model according to claim 2, wherein the second layer of cells line an opposing side of the membrane in the second microchannel.

4. The liver sinusoid model according to claim 1, further comprising a first means for providing continuous perfusion through the first microchannel and a second means for providing continuous perfusion through the second microchannel.

5. The liver sinusoid model according to claim 1, wherein the second layer of cells comprises a layer of liver sinusoidal endothelial cells.

6. The liver sinusoid model according to claim 5, wherein the second layer of cells is disposed on the first layer of cells, distal from the membrane.

7. The liver sinusoid model according to claim 1, wherein the first and second layers of cells comprise human cells.

8. The liver sinusoid model according to claim 1, wherein the first and second layers of cells comprise rat cells.

9. The liver sinusoid model according to claim 1, wherein the first and second layers of cells are viable for a time period greater than 30 days.

10. A liver sinusoid model comprising:
a generally planar substrate having first and second generally parallel microchannels formed therein, the first microchannel being disposed above the second microchannel;
a microporous membrane disposed between the first microchannel and the second microchannel, the microporous membrane having a first side and an opposing second side;
a layer of hepatocyte cells disposed in the first microchannel and extending directly along the first side of the microporous membrane; and
a layer of liver sinusoidal endothelial cells disposed in the second microchannel and extending along the second side of the microporous membrane.

11. A liver sinusoid bioreactor comprising:
a generally planar substrate having first and second generally parallel microchannels formed therein;
a microporous membrane disposed between and separating the first and second generally parallel microchannels;
a first layer of cells lining one side of the membrane in the first microchannel, wherein the first layer of cells are all a first common cell type;
a second layer of cells extending parallel to the first layer of cells in one of the first microchannel and the second microchannel, wherein the second layer of cells are all of a common cell type;
a first fluid circuit in fluid communication with the first microchannel, the first fluid circuit being adapted to flow an oxygenated fluid past the first layer of cells; and
a second fluid circuit in fluid communication with the second microchannel, the second fluid circuit being adapted to collect bile product secreted by the cells through the microporous membrane.

12. The liver sinusoid bioreactor according to claim 11, wherein the first layer of cells comprises a layer of hepatocyte cells.

13. The liver sinusoid bioreactor according to claim 11, wherein the first fluid circuit is adapted to flow in a first direction and wherein the second fluid circuit is adapted to flow in a second direction, opposite the first direction.

14. The liver sinusoid bioreactor according to claim 11, wherein the first layer of cells is disposed between the second layer of cells and the membrane.

15. The liver sinusoid bioreactor according to claim 14, further comprising a layer of collagen located between the first layer of cells and the second layer of cells.

16. The liver sinusoid bioreactor according to claim 11, wherein the second layer of cells is disposed on an opposing side of the membrane from the first layer of cells.

17. The liver sinusoid bioreactor according to claim 11, wherein the second layer of cells comprises a layer of liver sinusoidal endothelial cells.

18. The liver sinusoid bioreactor according to claim 11, wherein the first and second layers of cells comprise rat cells.

19. The liver sinusoid bioreactor according to claim 11, wherein the first and second layers of cells comprise human cells.

20. The liver sinusoid bioreactor according to claim 11, wherein the first and second layers of cells are viable for a time period greater than 30 days.
